(19) United States
(12) Patent Application Publication
(10) Pub. No.: US 2012/0123338 A1
(43) Pub. Date: May 17, 2012

(54) DAMPING SYSTEMS FOR STABILIZING MEDICATIONS IN DRUG DELIVERY DEVICES

(75) Inventors: Jeffrey P. Rouleau, Maple Grove, MN (US); Kenneth E. Rohly, Lino Lakes, MN (US); Timothy J. Denison, Minneapolis, MN (US)

(73) Assignee: MEDTRONIC, INC., Minneapolis, MN (US)

(21) Appl. No.: 13/387,071

(22) PCT Filed: Jun. 16, 2011
(86) PCT No.: PCT/US11/40753

§ 371 Date: Jan. 25, 2012

Related U.S. Application Data
(60) Provisional application No. 61/355,359, filed on Jun. 16, 2010.

(51) Int. Cl. A61M 5/142

(52) U.S. Cl. 604/151

(57) ABSTRACT

The invention relates to systems and methods for stabilizing therapeutic formulations used in medical devices. In particular, the invention relates to systems designed to inhibit the aggregation of therapeutic molecules such as polypeptides stored within a medication delivery device, for example polypeptide aggregation that can result from jostling the medication within a fluid medication reservoir. In embodiments of the invention, a damping system is operably connected to a fluid medication reservoir that is disposed within the housing of a device so as to inhibit the agitation of a fluid medication. Typically the damping system comprises one or more energy absorbing materials.

### CROSS REFERENCE TO RELATED APPLICATIONS

This application claims priority under Section 119(e) from U.S. Provisional Application Ser. No. 61/355,359, filed Jun. 16, 2010, the contents of which are incorporated herein by reference.

### FIELD OF THE INVENTION

The invention relates to systems and methods for stabilizing therapeutic formulations used in medical devices. In particular, the invention relates to systems designed to inhibit the destabilization of therapeutic molecules such as polypeptides stored within a fluid medication reservoir of a medication delivery device.

### BACKGROUND OF THE INVENTION

Therapeutic agents such as polypeptides can undergo numerous destabilizing physical and chemical changes over time that affect their potency and safety. Among these is aggregation, a phenomenon which includes dimerization, trimerization, and higher-order aggregates. Aggregation is a significant issue underlying multiple deleterious effects for peptide or protein-based therapeutics, including loss of efficacy, altered pharmacokinetics, reduced stability or product shelf life, and induction of unwanted immunogenicity. Aggregation can also have a dramatic effect on the bioavailability and pharmacokinetics of therapeutic polypeptides.

Therapeutic agents such as polypeptides disposed within a drug delivery device are commonly exposed to environmental conditions which can destabilize these agents, including for example vibrations and other mechanical stresses. Such environmental conditions can increase the occurrence of protein-protein interactions that favor aggregation. Insulin, interferon, and erythropoeitin are all observed to aggregate under conditions associated with their administration via drug delivery devices, an event which can elicit an immune response to these agents in a patient. Insulin can lose activity as a result of protein aggregation upon agitation at temperatures above those found in refrigerated storage. Therapeutic antibodies have similarly been shown to be subject to both inactivation and increased immunogenicity as a result of their aggregation.

Accordingly, there is a need for improved methods and systems that can stabilize therapeutic agents such as polypeptides in drug delivery devices.

### SUMMARY OF THE INVENTION

Embodiments of the invention disclosed herein comprise medical devices having damping systems designed to stabilize therapeutic formulations stored in such devices. Embodiments of the invention can be used, for example, to reduce mechanical stresses (e.g. those associated with accelerations and/or vibrations) that a medication is subjected to when a device such as a portable infusion pump is rocked, jostled or dropped. A typical embodiment of the invention is a medication infusion pump comprising a housing; a fluid medication reservoir disposed within the housing and adapted to store a medication to be administered to a user; and a damping system adapted to inhibit agitation of a medication stored within the fluid medication reservoir. In some embodiments of the invention, the damping system comprises one or more layers of a damping material (e.g. one that absorbs vibrations and/or other kinetic energies) disposed between the housing and the medication reservoir. In other embodiments of the invention, the damping system comprises one or more damping materials disposed over the housing. Certain embodiments of the invention include one or more layers of a damping material disposed between the housing and the medication reservoir in combination with one or more damping materials disposed over the housing. The damping material can comprise a polymeric material such as a rubber, a silicone, a polyurethane, a polyethylene, a polypropylene, a non conjugated diene, or combinations thereof. The damping material can also comprise a viscous fluid, either alone, or in combination with, a solid damping material. In certain embodiments of the invention, the damping material is further selected for an ability to insulate the fluid medication reservoir from fluctuations in environmental temperatures.

In some embodiments of the invention, the damping system comprises a fluid reservoir mount including one or more layers of a vibration absorbing material in operable contact with the medication reservoir. Optionally, the dampening system is part of a reservoir connector adapted to receive the drug reservoir and to fit within the housing in a manner that allows the reservoir connector system to be readily engaged and disengaged from the housing (e.g. to refill the fluid medication reservoir). Such receivable damping systems can for example, be disposed on a reservoir connector comprising elements such as a cap for coupling the fluid medication reservoir to a fluid conduit; a conduit cavity disposed in the cap and adapted to secure the fluid conduit to the cap; a housing engagement member adapted to engage the housing, wherein the reservoir connector is adapted to be releasably secured within the housing. Certain embodiments of the invention include one or more features designed to further stabilize a fluid medication stored within a delivery device. For example, in some embodiments of the invention, the fluid medication reservoir comprises a deformable container adapted to inhibit the formation of void spaces within the container as fluid is removed from the container. In embodiments of the invention, a fluid medication can comprise a polypeptide combined with an agent selected to inhibit the aggregation of the polypeptide (e.g. a surfactant).

The damping systems disclosed herein can be adapted for use with a variety of medical devices having various dimensions and geometries. In certain embodiments for example, the medication infusion pump comprises a continuous infusion pump having dimensions smaller than 15×15 centimeters (and typically smaller than 15×15×5 centimeters). Optionally, the medical device is operably coupled to an interface that facilitates its attachment to a user, wherein the interface comprises a clip, a strap, a snap, a clamp or an adhesive strip. In addition, embodiments of the invention can further include a sensing element such as a motion sensor and/or a temperature sensor that is operably coupled to the fluid medication reservoir so as to monitor the environmental conditions to which the fluid medication is exposed. Optionally the sensing element is coupled to an alarm that is activated in the event that the fluid medication is exposed to a mechanical stress or temperature that exceeds a critical threshold (e.g. a degree of mechanical stress or temperature threshold that can compromise the activity of a polypeptide medication).

A related embodiment of the invention is a medication infusion system comprising infusion set tubing; a medication infusion pump comprising: a housing; a fluid medication reservoir disposed within the housing and adapted to store a medication to be administered to a patient; and a damping system adapted to inhibit agitation of a fluid stored within the fluid medication reservoir. In this system, the pump includes a program memory; a processor for reading and executing the instructions contained in the program memory; and a motor adapted to deliver a fluid medication to a user. Typically the system further includes an interface that facilitates attachment of the pump to the user; and/or a reservoir connector that facilitates fluid medication reservoir insertion into and removal from the housing of the medication infusion pump. In such medication infusion systems, illustrative damping systems can comprise one or more layers of a vibration absorbing material disposed on the housing; and/or one or more layers of a vibration absorbing material disposed between the housing and the fluid medication reservoir, wherein at least one layer of material comprises a viscoelastic material for reducing accelerations experienced by a therapeutic agent that can result from movement of the housing. In certain embodiments of the invention, a damping system comprises elements that allow a user to modulate the extent of damping (e.g. one comprising an adjustable dashpot assembly). In some medication infusion systems of the invention, the damping system can further include an arrangement of elements designed to keep an ambulatory infusion device in a relatively level orientation while the device is in motion (e.g. a gimbal). Optionally in the medication infusion system, the fluid medication comprises a polypeptide such as an insulin or an interferon.

Yet another embodiment of the invention is a method of inhibiting aggregation of a polypeptide contained in a fluid medication reservoir within a medication infusion pump, wherein the pump is used to deliver a fluid medication to a user, the method comprising using a medication infusion pump comprising: a housing; a fluid medication reservoir disposed within the housing and adapted to store a medication to be administered to a patient; and a damping system adapted to inhibit agitation of a fluid stored within the fluid medication reservoir, wherein the inhibition of agitation inhibits the aggregation of a polypeptide contained in the fluid medication reservoir. Optionally in such methods, the damping system comprises one or more vibration absorbing materials disposed over the housing, wherein the vibration absorbing materials are adapted to decrease the magnitude of accelerative forces imparted on a medication disposed within the fluid medication reservoir.

In some embodiments of the invention, the damping system comprises a fluid reservoir mount including one or more layers of a vibration absorbing material. Optionally, the fluid reservoir mount comprises a reservoir connector including a cap for coupling the fluid medication reservoir to a fluid conduit; a conduit cavity disposed in the cap and adapted to secure the fluid conduit to the cap; a housing engagement member adapted to engage the housing; a tab disposed on the cap to provide a surface for a user to grip and twist the cap so as to engage the medication pump housing upon rotation of the cap. In certain embodiments of the invention, the damping materials are directly coupled to the reservoir connector. In other embodiments of the invention, the damping materials are directly coupled to the medication reservoir.

Other objects, features and advantages of the present invention will become apparent to those skilled in the art from the following detailed description. It is to be understood, however, that the detailed description and specific examples, while indicating some embodiments of the present invention are given by way of illustration and not limitation. Many changes and modifications within the scope of the present invention may be made without departing from the spirit thereof, and the invention includes all such modifications.

### BRIEF DESCRIPTION OF THE FIGURES

FIG. 1 provides a generalized diagram of an embodiment of a portable medication delivery system adapted for ambulatory use with a patient-user.

FIG. 2 provides generalized diagrams of damping systems comprising one or more damping materials disposed over the housing. FIG. 2A shows an embodiment of the invention where an infusion pump is completely enveloped by an outer soft wrap (e.g. one comprising a silicone or a low durometer polyurethane). FIG. 2B shows an embodiment of the invention where the outer wrap damping system includes a window to allow a user access to pump components. FIG. 2C shows an embodiment of the invention where the outer wrap damping system includes a conduit portal and a user attachment interface. FIG. 2D shows an embodiment of the invention where the outer wrap damping system includes a flap having a snap attachment element that can be used to secure the flap in a closed position.

FIG. 3 provides generalized diagrams of damping systems comprising one or more damping materials disposed between the housing and the medication reservoir. FIG. 3A shows an embodiment of the invention where a fluid medication reservoir is completely enveloped by a damping system that is coupled to the reservoir. FIG. 3B shows an embodiment of the invention where a fluid medication reservoir is partially enveloped by a damping system coupled to the reservoir connector.

FIG. 4 provides a graph of raw and reduced data obtained from accelerometers coupled to medication infusion pumps. In this figure the blue, green, and red plots are accelerometer data for the x, y, and z directions respectively, while the aqua color plot is the resultant acceleration magnitude calculated from the other three. The y-axis represents acceleration magnitude and the x-axis is the sample number (proportional to time).

FIG. 5 provides photographs of illustrative materials used to monitor fluid medication temperature. FIG. 5A shows an illustrative thermocouple data recorder. FIG. 5B shows an illustrative K-type thermocouple. Extension of yellow casing serves as spool for excess wire. FIG. 5C shows the recorder and thermocouple together.

FIG. 6 provides photographs of illustrative devices used to measure force. FIG. 6A shows an illustrative Ambulatory Data Recorder II (ADRII) unit. FIG. 6B shows an illustrative ADRII data recorder.

FIG. 7 provides a photograph of one of the composite devices used to generate an illustrative embodiment of the invention. A MiniMed 407C pump is shown below the thermocouple (upper) and ADRII (lower) devices.

FIG. 8 provides a graph of exemplary temperature data collected from one user over one week. The red line indicates the ambient temperature of the thermocouple recorder; the blue line indicates the temperature of the solution as sensed by the thermocouple.

### DETAILED DESCRIPTION OF THE INVENTION

Unless otherwise defined, all terms of art, notations and other scientific terms or terminology used herein are intended to have the meanings commonly understood by those of skill in the art to which this invention pertains. In some cases, terms commonly understood meanings are defined herein for clarity and/or for ready reference, and the inclusion of such definitions herein should not necessarily be construed to represent a substantial difference over what is generally understood in the art. As appropriate, procedures involving the use of commercially available kits and reagents are generally carried out in accordance with manufacturer defined protocols and/or parameters unless otherwise noted.

Certain chronic diseases may be treated, according to modern medical techniques, by delivering a medication or other substance to a patient's body, either in a continuous manner or at particular times or time intervals within an overall time period. For example, diabetes is a chronic disease that is commonly treated by delivering defined amounts of insulin to the patient at appropriate times. Some common modes of providing an insulin therapy to a patient include delivery of insulin through manually operated syringes and insulin pens. Other systems employ programmable pumps to deliver controlled amounts of insulin to a patient. In this context, embodiments of the present invention relate, generally, to delivery devices, systems and methods for delivering an infusion medium, such as one comprising a therapeutic polypeptide, to a recipient, such as a medical patient.

As noted above, in medication delivery devices such as portable infusion devices, therapeutic agents are exposed to a variety of environmental conditions that can compromise their therapeutic efficacy. These environmental conditions include fluctuating temperatures as well as mechanical agitation, phenomena which can lead to the aggregation of therapeutic polypeptides (see, e.g. Kwon et al., Pharm. Res. 2001, 18(2): 1754-1759; and Arakawa et al., Yakugaku Zasshi 2003, 123(11): 957-961). In this context, embodiments of the invention comprise a delivery device that includes one or more damping elements and/or systems configured to inhibit the aggregation of a therapeutic agent stored in a reservoir within the medical device (e.g. by reducing the agitating forces of accelerations that can occur when a device is jostled or dropped). The damping elements and systems described herein may be adapted for use with a wide variety of medication delivery devices known in the art such as the continuous infusion pump embodiments discussed in detail below.

While embodiments of the present invention are typically described herein with reference to continuous infusion pumps of the type that are commonly used, for example, for treating diabetes, other embodiments of the invention may be employed for delivering other infusion media to a patient-user for other purposes. For example, embodiments of the invention may be employed for delivering other types of drugs to treat diseases or medical conditions other than diabetes, including, but not limited to drugs for treating infections (e.g. interferon as used to treat hepatitis C infections) as well as the treatment of pain and certain types of cancers. Further embodiments may be employed for delivering other agents prone to destabilization/aggregation.

A typical embodiment of the invention is a medication infusion pump comprising a housing; a fluid medication reservoir disposed within the housing and adapted to store a medication to be administered to a user; and a damping system adapted to inhibit agitation of a medication stored within the fluid medication reservoir. In some embodiments of the invention, the damping system comprises one or more layers of a damping material disposed between the housing and the medication reservoir. In other embodiments of the invention, the damping system comprises one or more damping materials disposed over the housing, wherein the materials are adapted to decrease the magnitude of accelerative force imparted on a medication disposed within the fluid medication reservoir. Certain embodiments of the invention include one or more layers of a damping material disposed between the housing and the medication reservoir in combination with one or more damping materials disposed over the housing. The damping materials used in embodiments of the invention typically comprise materials selected for their ability to absorb mechanical kinetic energy in the form of vibrations and the like. In certain embodiments of the invention, the material is further selected for an ability to insulate the fluid medication reservoir from fluctuations in environmental temperature that can be experienced by a medication infusion pump.

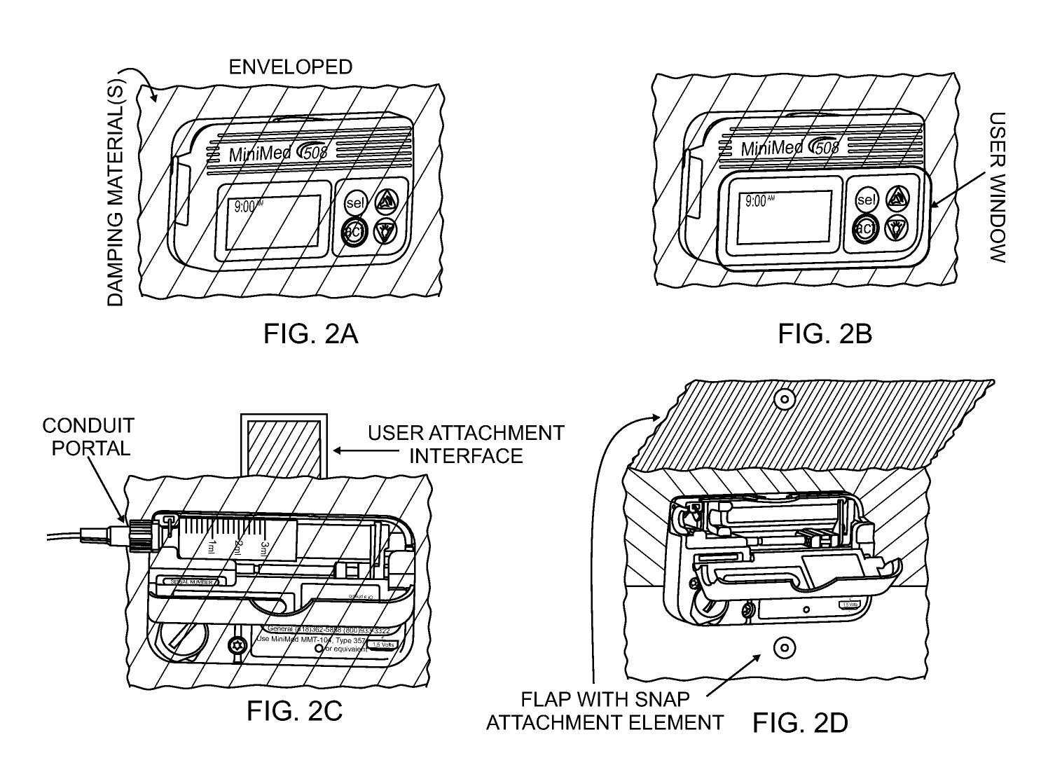

In some embodiments of the invention, one or more damping materials is disposed over the housing. FIG. 2 provides generalized diagrams of damping systems comprising one or more damping materials disposed over the housing. FIG. 2A shows an embodiment of the invention where an infusion pump is completely enveloped by an outer soft wrap (e.g. one comprising a silicone or a low durometer polyurethane). FIG. 2B shows an embodiment of the invention where the outer wrap damping system includes a window to allow a user access to pump components. FIG. 2C shows an embodiment of the invention where the outer wrap damping system includes a conduit portal and a user attachment interface. FIG. 2D shows an embodiment of the invention where the outer wrap damping system includes a flap having a snap attachment element that can be used to secure the flap in a closed position.

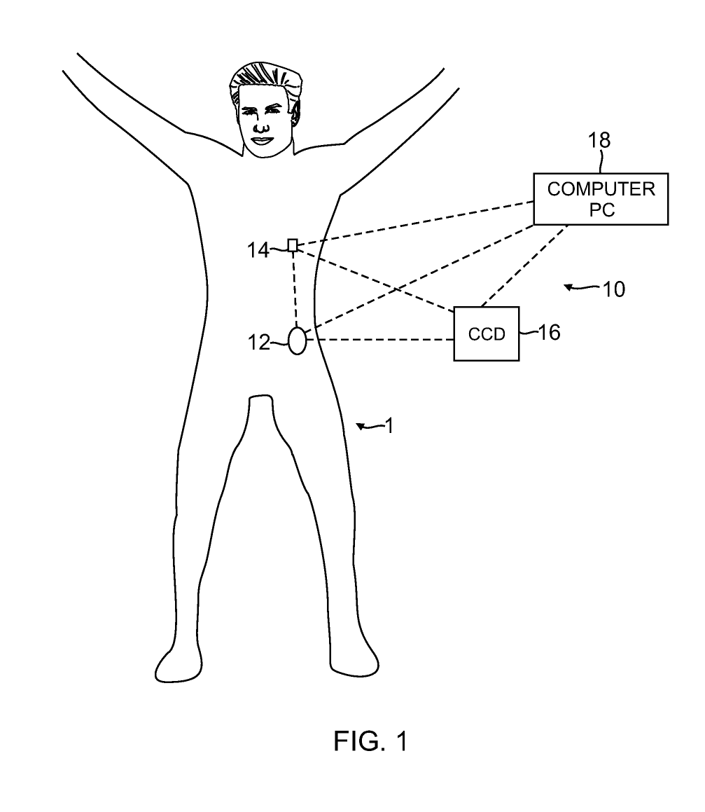

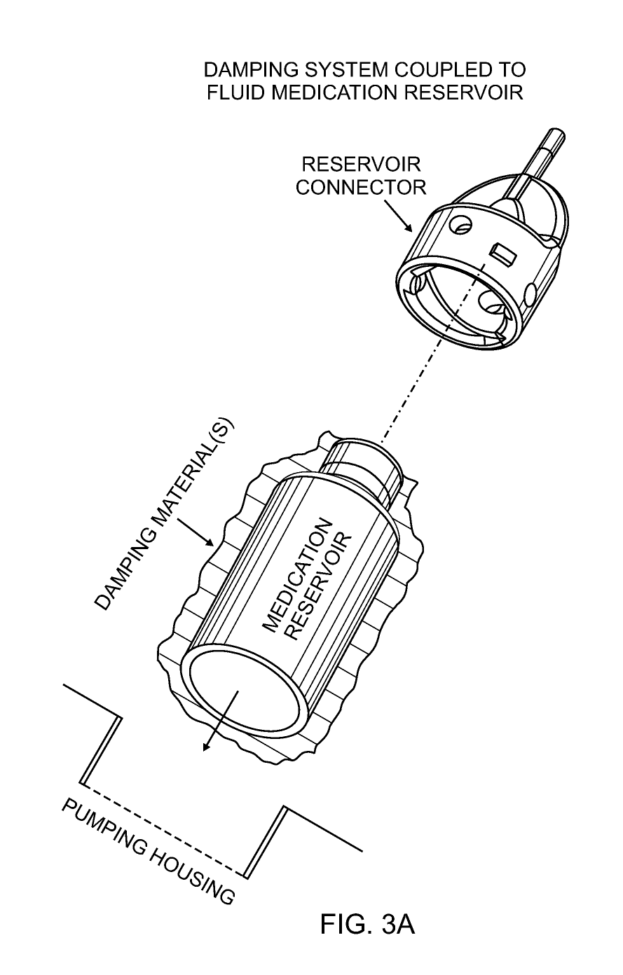

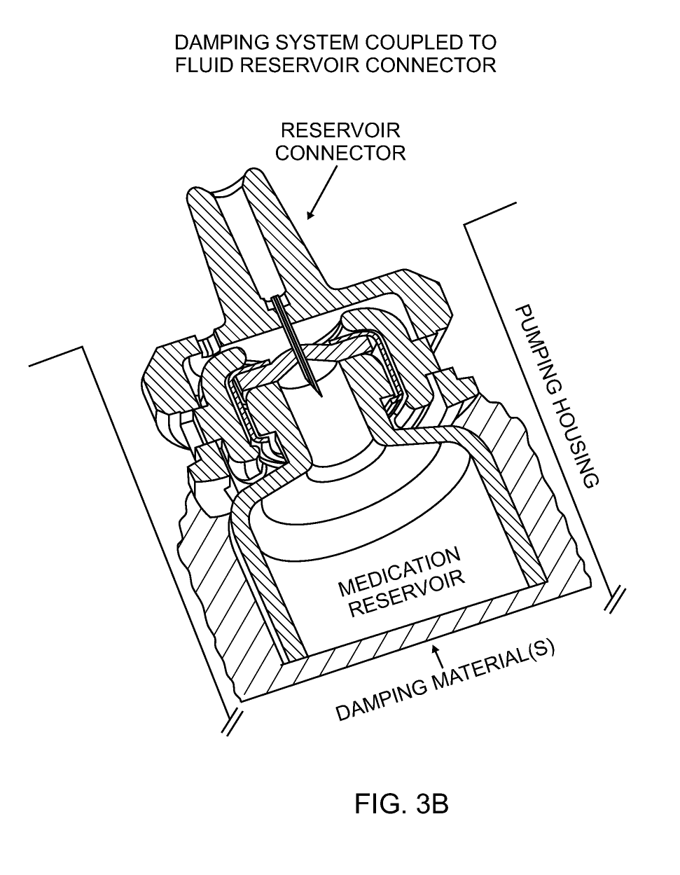

In other embodiments of the invention, the damping system comprises a fluid reservoir mount including one or more layers of a vibration absorbing material in operable contact with the medication reservoir. Optionally, the dampening system is adapted to receive the drug reservoir and to fit within the housing in a manner that allows the system to be readily engaged and disengaged from the housing (e.g. to refill the fluid medication reservoir). For example in some embodiments of the invention, a receivable damping system is part of a reservoir connector such as those disclosed for example in U.S. Pat. No. 7,658,734 (see, e.g. the reservoir connector embodiments shown in FIGS. 1, 3, 5 and 12), the contents of which are incorporated by reference herein. In one illustrative embodiment, the damping system is disposed on a reservoir connector comprising a cap for coupling the fluid medication reservoir to a fluid conduit; a conduit cavity disposed in the cap and adapted to secure the fluid conduit to the cap; a reservoir engagement element adapted to engage the fluid reservoir, a housing engagement member adapted to engage the housing, wherein the reservoir connector is adapted to be releasably secured within the housing. FIG. 3 provides generalized diagrams of damping systems comprising one or more damping materials disposed between the housing and the medication reservoir. FIG. 3A shows an embodiment of the invention where a fluid medication reservoir is completely enveloped by a damping system that is coupled to the reservoir. FIG. 3B shows an embodiment of the invention where a fluid medication reservoir is partially enveloped by a damping system that is coupled to the reservoir connector.

As noted above, embodiments of the invention comprise damping systems designed to stabilize therapeutic agents such as polypeptides prone to aggregation. As is known in the art, damping is the dissipation of energy, for example by releasing it in the form of low-grade heat. In this context, the embodiments of the invention disclosed herein can employ, either alone or in combination, a wide variety of damping methods, materials and architectures/configurations known in the art. For example, embodiments of the invention can employ dry friction in damping, a common damping mechanism, and the reason an object sliding on a surface will slow down and stop. Alternatively, embodiments of the invention can employ viscous damping as a means of energy dissipation. Alternatively, embodiments of the invention can employ hysteretic damping to dissipate energy.

Typically, one or more layers of a damping material is used in embodiments of the invention disclosed herein. A wide variety of suitable damping materials that absorb vibration energy are known in the art and include, for example, silicone, low durometer polyurethane and other polymers such as soft vinyl chloride resins formed of a vinyl chloride resin added with a plasticizer. Damping materials can be designed so as to attenuate the vibration energy by consuming the vibration energy in the material as frictional heat. Rubber materials such as butyl rubber and acrylonitrile-butadiene rubber can also be used as vibration damping materials, which are excellent in terms of processability, mechanical strength, and costs. Compositions formed mainly of a polymeric material and a piezoelectric powdery material are disclosed in JP 03-188165 A, and Inaba, et al., “Relationship between Mechanical Properties and Damping Performance of Piezoelectric Damping composites,” Journal of The Society of Rubber Industry, Japan, vol. 67, p. 564 (1994). Such compositions can absorb and attenuate vibrations by converting vibration energy into electric energy by the action of electro-mechanical conversion of the piezoelectric material and dispersing the electric energy.

Layered structures including elastomeric materials that absorb energy are useful as damping materials. Optionally, the damping (e.g. vibration absorbing) material comprises a polymeric material such as a silicone, a polyurethane (e.g. a low durometer polyurethane), a rubber, a polyethylene, a polypropylene, a non-conjugated diene, or combinations thereof. In certain embodiments of the invention, a damping material comprises a composite, such as a composite of a metal and a polymer. Some damping systems can employ an elastomer in combination with a glassy polymer, metal, or combination thereof which are in contrast to single phase homogenous materials, i.e. monolithic systems.

In embodiments of the invention, the skilled artisan can select a material having appropriate properties for the application in which it is to be used. For example, in certain embodiments of the invention, materials used for damping can be those selected for their ability to exhibit large viscous losses in response to deformation. As is known in the art, such losses can be characterized or quantified, for example, in terms of, for example, dynamic Young's moduli or dynamic shear moduli. The dynamic storage modulus, by definition, is proportional to the amplitude of the stress which results in response to a sinusoidal strain applied in phase with the stress (where the strain may be either shear or elongational depending on whether shear or Young's modulus is desired respectively). Similarly, the loss modulus is, by definition, proportional to the amplitude of the stress which results in response to the application of a sinusoidal strain rate applied out of phase with the stress.

In this context, in certain embodiments of the invention, a material used in a damping system is selected to have desirable Young's moduli or dynamic shear moduli properties that are at least equal to that of one of the illustrative materials disclosed herein, for example a low durometer polyurethane (or a silicone or a rubber etc.). In one illustrative embodiment, the material comprises a low-Durometer urethane polymer which comprises a urethane having a hardness of less than 40 Shore A on the Durometer A scale (see, e.g. the American Society for Testing and Materials D2240-00 testing standard). A variety of known urethane polymers may be formulated to yield hardness in this range, with the ratio of components adjusted to achieve a precise hardness. For example, in one embodiment of the viscoelastic dampening system, the system is constructed of a material having damping properties that are at least equal to a Variable Hardness Polyurethane® formulated to have 10 Shore A Durometer hardness. Variable Hardness Polyurethane® is commercially available from Crosslink Technology Inc., and is constructed from a mixture of Diphenyl methane Diisocyanate (MDI) and polyglycol blends. The material further comprises 15-40% Dibutyl Phthalate and 15-40% Di-(Methyl-Thio) toluenediamine. In another illustrative embodiment of the viscoelastic dampening system, the system is constructed of a material having damping properties that are at least equal to a low-Durometer urethane of approximately 30 Shore A hardness comprising Toluenediisocyanate (TDI) polyester glycol pre-polymer containing 45 parts per hundred of a phthalate plasticizer and 60 parts per hundred of a silica filler, combined with a multi functional glycol/catalyst blend.

In addition to the polymeric materials such as those discussed above, certain embodiments of the invention can use viscous fluids as damping materials (e.g. either alone, or in combination with a polymeric material). Viscous fluid materials can be utilized, for example, to damp out vibrations from a vibrating body in instances where the vibrations may not be reduced sufficiently with solid damping materials. In this context, a variety of viscous fluid materials and associated mounts are known in the art. Typical embodiments of the invention that utilize such viscous fluids include a viscous fluid container system that is operably coupled (e.g. encloses at least a portion) to the fluid medication reservoir. In some embodiments of the invention, a viscous fluid mount is used as an interconnection between a vibrating body (e.g. a user, a medical device attached the user etc.) and a fluid medication reservoir disposed within a medical device. The viscous fluid mount can include, for example, a chamber, a high viscosity damping fluid contained within the chamber and a flow resistance element. In this arrangement, input energy from a vibrating body is dissipated by shear friction of the damping fluid moving parallel to the shear surface area of the flow resistance element. Optionally the viscous fluid mount and/or the viscous fluid chamber is coupled to a reservoir connector that is adapted to receive the fluid medication reservoir, wherein the reservoir connector is adapted to engage and disengage the medication device in order to, for example, facilitate the filling/refilling of the fluid medication reservoir.

A variety of viscous fluid damping systems known in the art can be adapted for use with embodiments of the invention. In one illustrative example, a viscous fluid damping system includes a housing having an inner cylinder and an outer cylinder which disposed concentrically with each other. In this arrangement, an annular space formed between the inner cylinder and the outer cylinder contains a viscous fluid and a cylindrical rotor accommodated in the housing can receive a damping action from the viscous fluid. Other viscous fluid damping systems can include a housing which has an annular space containing a viscous fluid, a cap, a cylindrical rotor, a ring-shaped first sealing member disposed between the rotor and the cap, and, optionally, another ring-shaped sealing member disposed between the rotor and the housing. U.S. Pat. Nos. 5,979,882; 5,855,259; 5,880,410; 5,232,062; and 6,662,683 disclose further illustrative non-limiting viscous fluid damping system elements and/or assemblies known in the art that can be adapted for use with embodiments of the invention.

A variety of damping configurations can be used in embodiments of the invention. For example, embodiments of the invention can comprise free or unconstrained layer damping. One such embodiment comprises a layer of an appropriate damping material coupled to those surfaces of the structure which are vibrating (e.g. the surface of a medication reservoir). In such systems, damping can result from cyclic tension/compression deformation of the material, resulting in dissipation of energy. Embodiments of the invention can employ a single constrained damping layer in the form of a thin layer of damping material combined with a constraining layer of metallic foil or similar rigid material. The damping mechanism with such single constrained damping layer typically involves cyclic shear deformation of the damping material.

Embodiments of the invention can comprise, for example, a constrained layer vibration damper in the form of a layered laminate. Optionally the laminate comprises a meltable bonding layer to be brought into contact with an element to be damped (e.g. a fluid medication reservoir), a viscoelastic layer and a constraining layer laminated such that the viscoelastic layer is intimately sandwiched between the bonding layer and the constraining layer. The constraining layer of this damper can be formed of a resin composition. The viscoelastic layer can be formed of a viscoelastic and adhesive material, and serve the function of damping or attenuating mechanical vibrations. For example, U.S. Pat. No. 5,407,034 discloses a damping structure including damping layers of viscoelastic material alternating with an outer metal layer, a middle metal layer and an inner metal layer.

Certain embodiments of the invention can comprise a dashpot assembly, a mechanical damping device configuration comprising a piston that moves through a viscous material (e.g. a gas or a fluid). Optionally, the fluid or gas pressure within the dashpot assembly can be adjusted in order to control the compression or damping of the dashpot shock absorption. A variety of such mechanisms are known in the art which can be adapted for use with embodiments of the invention. For example, U.S. Pat. No. 7,699,147 teaches an adjustable dashpot with shock-absorption force controls, with at least one flow-regulating system including one or more shock-absorption components for the compression phase and/or for the decompression phase so as to allow the dashpot to shift continuously between the hard and soft phases (see, e.g. the embodiment disclosed in FIG. 1). U.S. Pat. No. 7,219,881 similarly discloses an adjustable dashpot shock absorber having selectable behavior characteristic. In this system, the gas pressure within a chamber of the device can be adjusted via a valve in order to set a basic strength for the compression or damping of the shock absorber (see, e.g. column 1).

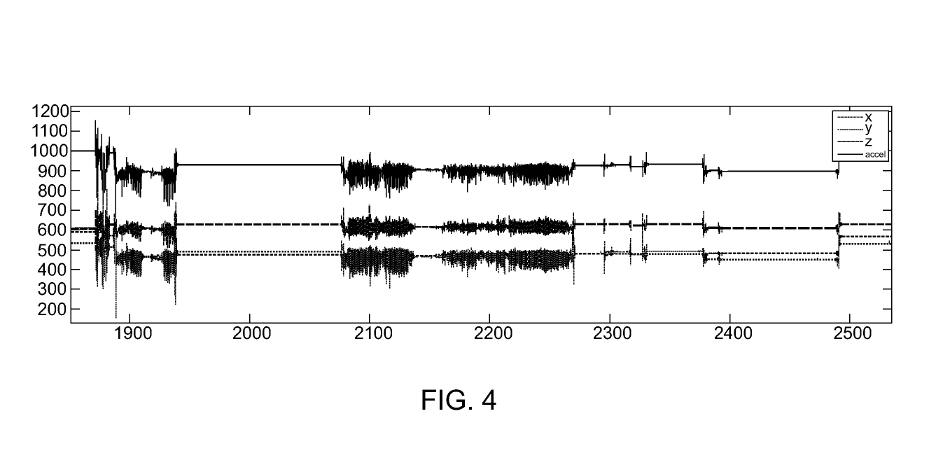

Some embodiments of the invention can further include a sensing element such as a motion sensor and/or a temperature sensor that is operably coupled to the fluid medication reservoir so as to monitor the environmental conditions to which the fluid medication is exposed. Optionally such sensing elements are operably connected to the housing of a medical device that also comprises the fluid medication reservoir, for example the housing of an insulin infusion pump. In one illustrative embodiment of the invention, the sensor comprises an accelerometer which measures the mechanical stresses (e.g. those associated with accelerations and/or vibrations) that a medication is subjected to when a device such as a portable infusion pump is rocked, jostled or dropped. While there are several mechanisms for measuring force that are commonly employed by accelerometers, nearly all are electrical in nature, that is the forces experienced by the device are linked to a change in generated voltage, capacitance, or resistance. While an individual accelerometer can measure forces in one direction, the linking of multiple devices and compilation of their data can provide information in multiple dimensions. These multi-directional systems have become popular in handheld video game controllers; in automobiles to detect skids and deploy braking systems, active suspension control systems, and airbags; and many other motion-sensitive devices. In this context, a number of accelerometers are known in the art which can be adapted for use with embodiments of the invention. For example, U.S. Patent Application No. 2007/0208530 discloses an activity monitor comprising a housing for attachment to a person; at least one accelerometer disposed within the housing; and a processor disposed within the housing, for processing signals from the accelerometer to assess activity of the person (see, e.g. paragraphs [0089]-[0091]). FIG. 4 provides a graph of raw and reduced data obtained from accelerometers coupled to medication infusion pumps.

A wide variety of temperature sensor systems are known in the art that can be adapted for use with embodiments of the invention, for example to monitor the temperature of a fluid medication so as to ensure that the medication is not exposed to excessive high (or low) temperatures that have the potential to compromise the activity of a medication such as insulin. Temperatures can be measured by many methods. Electrical methods of temperature measurement are extremely sensitive and convenient due to their easy interface with computers for data collection and analysis. Temperature observations can be made based on: 1) generated voltage, 2) changes in capacitance, or 3) changes in resistivity. A common temperature-sensing device is a thermocouple, which relies on generated voltage. A thermocouple measures the temperature-dependent variation in the voltage generated by the interface of two dissimilar metals; two metal wires are connected at both ends and one end is exposed to a variation in temperature. If the circuit is broken in the center, the voltage becomes a function of the types of metals and their temperature. Thermocouples are convenient in that they do not require a power source or battery, they are sensitive to a wide range of temperatures, and they can be immersed in a fluid without adverse effects. Thermocouples are named based on the type of metals used. In one illustrative embodiment of the invention, a K-type thermocouple with a chromel-alumel wire is used.

The Omega OM-CP-RFTC4000A device that is used in Example 1 below provides a miniature, wireless, battery powered, stand-alone, thermocouple based temperature transmitter. The wireless transmitter in this sensor can transmit readings back to the host computer where the data can be analyzed in real time and logged to the device's memory. U.S. Pat. No. 7,704,227 teaches an infusion pump that includes a temperature sensor for monitoring temperatures such as the threshold temperature at which as medication such as insulin can degrade and become less effective (see, e.g. column 8). Similarly, U.S. Pat. No. 6,302,855 teaches a medication delivery device that includes a sensor to monitor the temperature of a medication contained inside the device and that is designed for use with medications sensitive to temperature that can be ruined or damaged if the temperature of the medication is outside a given interval (see, e.g. column 2).

In some embodiments of the invention, the sensor is operably coupled to data storage element(s) and/or data processing element(s) that allows a user to maintain a record of the mechanical and/or thermal stresses to which a medication is exposed. For example, U.S. 20070229248 discloses a sensor tag that can be fixed on to or near to a mechanical or electronic system that comprises a sensor in the form of an accelerometer or strain gauge, a calculating means to receive signals from the sensor and to calculate the resulting damage, a memory to record data representing the resulting and relative damage, and means to enable the recorded data to be read (see, e.g. paragraphs [0021]-[0028]). Similarly, U.S. Pat. No. 6,605,038 discloses an accelerometer worn on the upper arm that is coupled to a central monitoring unit generates analytical status data that is transmitted to a recipient (see, e.g. column 22).

Some embodiments of the invention can include a sensing element coupled to an alarm that is activated in the event that the fluid medication is exposed to a mechanical stress or temperature that exceeds a critical threshold (e.g. a degree of mechanical stress or temperature threshold that can compromise the activity of a polypeptide medication). Such mechanical stress or temperature alarms can be distinguishable from each other and can include any one of a wide variety of signals such as audible signals, visual signals, tactile signals, displays, and/or the like. In one illustrative embodiment, the alarm comprises a visual indicator that changes color when the sensor detects a mechanical stress or temperature that exceeds a critical threshold value (e.g. a temperature below 4° C., a temperature above 40° C. etc.).

In certain embodiments of the invention, the fluid medication reservoir comprises additional elements designed to inhibit polypeptide aggregation, for example a deformable container adapted to inhibit the formation of void spaces within the container as fluid is removed from the container. In some embodiments of the invention, a polypeptide medication is disposed within the fluid medication reservoir, wherein the fluid medication is combined with an agent selected to inhibit the aggregation of the polypeptide, for example a surfactant such as a Genapol®. In certain embodiments, the medication infusion pump comprises a continuous infusion pump having dimensions smaller than 15×15 centimeters (and typically smaller than 15×15×5 centimeters).

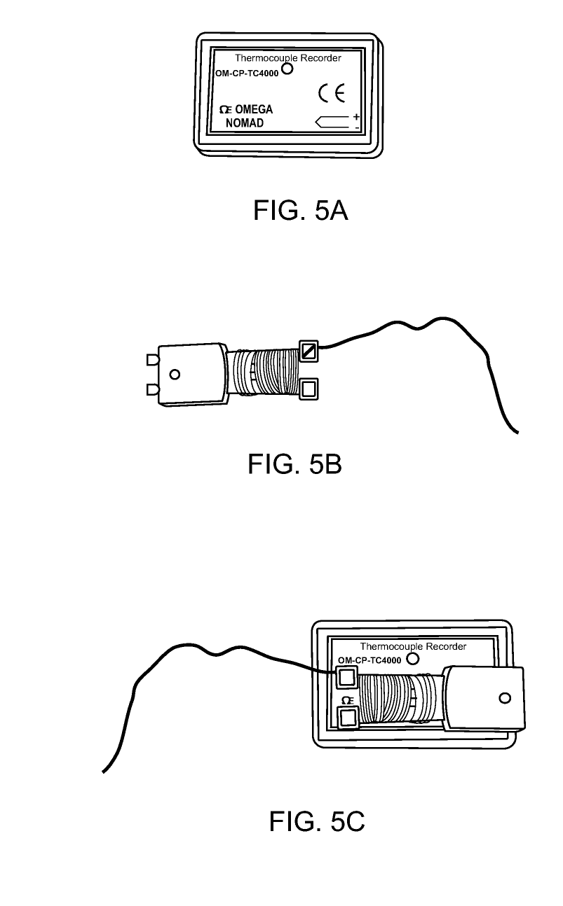

Optionally, the medical device is operably coupled to an interface that facilitates its attachment to a user. For example, in some embodiments of the invention, the interface can comprise a clip, a strap, a snap, a clamp or an adhesive strip. In embodiments of the invention, an element of the device such as the interface can comprise a gimbal, i.e. an arrangement of elements that is designed to keep equipment such as portable infusion device in a certain orientation despite the motion of the user (e.g. so as to inhibit the shaking of the device elements). In some embodiments of the invention, other elements of the device such as the assembly that holds the fluid medication reservoir within the device can comprise a gimbal. A variety of gimbal mounts/systems are known in the art can be adapted for use with embodiments of the invention disclosed herein. For example, U.S. Patent Application 20040008157 (the contents of which are incorporated by reference herein), discloses a portable gimbal assembly that is designed to be attached to a baseball cap visor or hat brim (see, e.g. the embodiment disclosed in FIG. 1). U.S. Pat. No. 6,817,974 (the contents of which are incorporated by reference herein), discloses a number gimbal systems designed for use with medical device elements including cables (see, e.g. the embodiment disclosed in FIG. 5). In addition, U.S. Pat. Nos. 5,978,137, 5,579,071, and 6,831,765 and U.S. Patent Applications 20080115611, 20060053912 and 20090052037 also disclose a number of illustrative non-limiting gimbal system elements and/or assemblies known in the art.

A related embodiment of the invention is a medication infusion system comprising infusion set tubing; a medication infusion pump comprising: a housing; a fluid medication reservoir disposed within the housing and adapted to store a medication to be administered to a patient; and a damping system adapted to inhibit agitation of a fluid stored within the fluid medication reservoir. The pump includes a program memory; a processor for reading and executing the instructions contained in the program memory; and a motor adapted to deliver a fluid medication to a user. Typically the system further includes an interface that facilitates attachment of the pump to the user; and/or a reservoir connector that facilitates fluid medication reservoir insertion into and removal from the housing of the medication infusion pump. In such systems, an illustrative damping system comprises one or more layers of a vibration absorbing material disposed on the housing; and/or one or more layers of a vibration absorbing material disposed between the housing and the fluid medication reservoir, wherein at least one layer of material comprises a viscoelastic material for reducing accelerations to the pump that result from movement of the housing. In some medication infusion systems of the invention, the damping system includes a fluid reservoir mount comprising a gimbal; and/or the interface that facilitates attachment of the pump to the user comprises a gimbal. Optionally in the medication infusion system, the fluid medication reservoir comprises an insulin or an interferon.

As noted above, embodiments of the invention include infusion devices comprising elements and/or arrangements of elements adapted to reduce the magnitude of the various forces that can act on (e.g. agitate) a fluid medication contained within these devices (e.g. vibrations and the like). Embodiments of the invention include devices having a first layer of a damping material operatively coupled to (e.g. disposed around) a fluid medication vial. Optionally this first layer is operatively coupled to a second layer of a damping material, which can optionally be coupled a third layer (or fourth layer) of a damping material etc. These material layers can be of various dimensions, for example having a thickness greater than (or less than) 1, 2, 3, 4, 5, 6, 7, 8, 9 or 10 mm. In certain embodiments of the invention, a material is selected to have desirable Young's moduli or dynamic shear moduli properties that are at least equal to that of one of the illustrative materials disclosed herein, for example a silicone, a polyurethane (e.g. a low durometer polyurethane), a rubber, a polyethylene, a polypropylene, a non-conjugated diene, or combinations thereof.

Embodiments of the invention also include methods of damping using the damping systems disclosed herein. One such embodiment of the invention is a method of inhibiting aggregation of a polypeptide contained in a fluid medication reservoir within a medication infusion pump, wherein the pump is used to deliver a fluid medication to a user, the method comprising using a medication infusion pump comprising: a housing; a fluid medication reservoir disposed within the housing and adapted to store a medication to be administered to a patient; and a damping system adapted to inhibit agitation of a fluid stored within the fluid medication reservoir, wherein the inhibition of agitation inhibits the aggregation of a polypeptide contained in the fluid medication reservoir. Optionally in such methods, the damping system comprises one or more vibration absorbing materials disposed over the housing, wherein the vibration absorbing materials are adapted to decrease the magnitude of accelerative force imparted on a medication disposed within the fluid medication reservoir.

In some embodiments of the invention, the damping system comprises a fluid reservoir mount including one or more layers of a vibration absorbing material. Optionally, the fluid reservoir mount comprises a cap for coupling the fluid medication reservoir to a fluid conduit; a conduit cavity disposed in the cap and adapted to secure the fluid conduit to the cap; a housing engagement member adapted to engage the housing; a tab disposed on the cap to provide a surface for a user to grip and twist the cap so as to engage the medication pump housing upon rotation of the cap. Some embodiments of the invention further comprise using a fluid medication reservoir comprising a deformable container adapted to inhibit the formation of void spaces within the container as fluid is removed from the container.

As noted above, in certain embodiments of the invention, the therapeutic agent is administered in a “substantially continuous manner” via a medical device having a damping system as disclosed herein. Typically the therapeutic agent is administered in a substantially continuous manner via a continuous infusion pump, for example a pump typically used to administer insulin to a diabetic patient. Suitable types of pumps that can employ damping systems include, but are not limited to, osmotic pumps, interbody pumps, infusion pumps, implantable pumps, peristaltic pumps, other pharmaceutical pumps, or a system administered by insertion of a catheter at or near an intended delivery site, the catheter being operably connected to a pharmaceutical delivery pump. It is understood that such pumps can be implanted internally (e.g. into a patient's abdominal (peritoneal) cavity) or worn externally (e.g. clipped to belt loop) as appropriate. Typical methods of the invention employ a programmable pump for the methods described herein.

An example of an external pump is Medtronic MiniMed® pump and one example of a suitable implantable pump is Medtronic SynchroMed® pump, both manufactured by Medtronic, Minneapolis, Minn. In these pumps, the therapeutic agent is pumped from the pump chamber and into a drug delivery device, which directs the therapeutic agent to the target site. The rate of delivery of the therapeutic agent from the pump is typically controlled by a processor according to instructions received from the programmer. This allows the pump to be used to deliver similar or different amounts of the therapeutic agent continuously, at specific times, or at set intervals between deliveries, thereby controlling the release rates to correspond with the desired targeted release rates. A therapeutic polypeptide may be delivered subcutaneously, intramuscularly, parenterally, intraperitoneally, transdermally, or systemically. In specific embodiments, a therapeutic polypeptide may be delivered subcutaneously or for a systemic infusion. A drug delivery device may be connected to the pump and tunneled under the skin to the intended delivery site in the body. Suitable drug delivery devices include, but are not limited to, those devices disclosed in U.S. Pat. Nos. 6,551,290 and 7,153,292.

A wide variety of continuous infusion devices known in the art can incorporate a damping system and deliver one or more therapeutic agents to a patient infected with a virus such as HCV, a patient suffering from a cancer such as melanoma, or a patient suffering from a metabolic disorder such as diabetes. Continuous administration may for example be accomplished using an infusion pump for the subcutaneous or intravenous injection at appropriate intervals, e.g. at least hourly, for an appropriate period of time in an amount which will facilitate or promote a desired therapeutic effect. Typically the continuous infusion device used in the methods of the invention has the highly desirably characteristics that are found for example in pumps produced and sold by the Medtronic corporation. In illustrative embodiments of the invention, the cytokine is administered via an infusion pump such as a Medtronic MiniMed model 407C, model 508, or another infusion pump made by the Medtronic MiniMed Diabetes division, Northridge Calif. Typically the pump includes a small, hand-held remote programmer, which enables diabetes patients to program cytokine delivery without accessing the pump itself.

Alternatively, continuous administration can by accomplished by, for example, another device known in the art such as a pulsatile electronic syringe driver (Provider Model PA 3000, PANCRETEC Inc., San Diego Calif.), a portable syringe pump such as the Graseby model MS 1 6A (GRASEBY MEDICAL Ltd., Watford, Herts England), or a constant infusion pump such as the DISETRONIC MODEL PANOMAT C-S OSMOTIC PUMP as available from ALZA, may also be used. Since use of continuous subcutaneous injections allows the patient to be ambulatory, it is typically chosen for use over continuous intravenous injections.

Infusion pumps and monitors for use in embodiments of the invention can be designed to be compact (e.g. less than 15×15 centimeters, less than 15×15×5 centimeters etc.) as well as water resistant, and may thus be adapted to be carried by the user, for example, by means of a belt clip. As a result, important medication can be delivered to the user with precision and in an automated manner, without significant restriction on the user's mobility or life-style. The compact and portable nature of the pump and/or monitor in combination with a damping system as disclosed herein affords a high degree of versatility in using the device. As a result, the ideal arrangement of the pump can vary widely, depending upon the user's size, activities, physical handicaps and/or personal preferences. In a specific embodiment, the pump includes an interface that facilitates the portability of the pump (e.g. by facilitating coupling to an ambulatory user). Typical interfaces include a clip, a strap, a clamp or a tape. Optionally the interface further comprises a gimbal.

One illustrative embodiment of an infusion medium delivery system 10 which can include the damping elements and/or systems disclosed herein is shown in FIG. 1. Typically, such systems include a delivery device 12 including a damping element of the invention described herein. The system 10 may also include other components known in the art. For example, the system can be coupled for communication with the delivery device 12, including, but not limited to, a sensor or monitor 14, a command control device (CCD) 16 and a computer 18. Each of the CCD 16, the sensor or monitor 14, the computer 18 and the delivery device 12 may include receiver, transmitter or transceiver electronics that allow communication with other components of the system. The delivery device 12 may include electronics and software for analyzing sensor data and for delivering the infusion medium according to sensed data and/or pre-programmed delivery routines. Some of the processing, delivery routine storage and control functions may be carried out by the CCD 16 and/or the computer 18, to allow the delivery device 12 to be made with more simplified electronics. However, in other embodiments, the system 10 may include a delivery device 12 that operates without any one or more of the other components of the system 10 shown in FIG. 1. Examples of the types of communications and/or control capabilities, as well as device feature sets and/or program options may be found in U.S. patent application Ser. No. 10/445,477 filed May 27, 2003, and entitled “External Infusion Device with Remote Programming, Bolus Estimator and/or Vibration Alarm Capabilities,” and U.S. patent application Ser. No. 10/429,385 filed May 5, 2003, and entitled “Handheld Personal Data Assistant (PDA) with a Medical Device and Method of Using the Same,” U.S. patent application Ser. No. 09/813,660 filed Mar. 21, 2001, and entitled “Control Tabs For Infusion Devices And Methods Of Using The Same”.

In the generalized system diagram of FIG. 1, the delivery device 12 and sensor or monitor 14 are secured to a patient-user 1. The locations at which those components are secured to the patient-user 1 in FIG. 1 are provided only as a representative, non-limiting example. The delivery device 12 and sensor or monitor 14 may be secured at other locations on the patient-user 1, and such locations may depend upon the type of treatment to be administered by the system 10. Such other locations may include, but are not limited to, other locations on the patient-user's body, locations on the patient-user's clothing, belt, suspenders, straps, purse, tote or other structure that may be carried by the patient-user. As described in further detail below, the delivery device 12 contains a reservoir of an infusion medium and a pump, for delivering the infusion medium, such as, but not limited to an insulin formulation, into the patient-user's body in a controlled manner. Control instructions and/or data may be communicated between the delivery device 12, the sensor or monitor 14, the CCD 16 and the computer 18. The delivery device 12 may be configured to secure to the skin of a patient-user 1, in the manner of a patch, at a desired location on the patient-user. In such embodiments, it is desirable that the delivery device 12 have relatively small dimensions for comfort and ability to conceal the device, for example, under a garment.

Embodiments of the invention are adapted for use with a variety of other devices known in the art, for example patch-like delivery devices as well as implantable delivery devices. Examples of patch-like delivery devices are described in U.S. patent application Ser. No. 11/211,095, filed Aug. 23, 2005. Delivery devices described in U.S. patent application Ser. No. 11/211,095 employ a reservoir structure having a moveable plunger for selectively driving fluid from the reservoir. Examples of implantable devices that can include the damping systems disclosed herein include those described in U.S. Pat. No. 7,288,085. U.S. Pat. No. 7,288,085 describes an implantable medication delivery device with a permanent magnet solenoid pump that comprises a housing, a fluid medication reservoir, a power source, electronics, and a permanent magnet solenoid pump. Typically, the therapeutic substance delivery device components are carried in a single housing that is manufactured from a material that is biocompatible and hermetically sealed such as titanium, tantalum, stainless steel, plastic, ceramic, and the like. The therapeutic substance reservoir can be placed inside the housing or can be separated from the housing with a fluid coupling such as a tube between the reservoir and the housing. The therapeutic substance reservoir can be configured to contain a therapeutic substance such as and may use geometries that are complementary with a damping system as disclosed herein, such as a bellows, collapsible bag, and the like.

Embodiments of the invention include a fluid medication reservoir disposed within the housing of a medical device and operable coupled to a damping system as disclosed herein. Typically, the reservoir includes a container having an internal volume for containing a fluidic infusion medium, such as, but not limited to an insulin formulation. The reservoir may be made of any material suitably compatible with the infusion medium, including, but not limited to suitable metal, plastic, ceramic, glass, composite material or the like. For example, the reservoir may be formed of a plastic material referred to as TOPAS (trademark of Ticona, a subsidiary of Celanese Corporation), such as described in U.S. patent application Ser. No. 11/100,188, filed Apr. 5, 2005 (Publication No. 2005/0197626)).

In embodiments of the invention, the reservoir and/or damping system may be supported by a reservoir retaining portion of the housing in any suitable manner. For example, the reservoir may be supported on a surface of a matrix (e.g. the wall of a housing, a reservoir connector, etc.) and held in place by one or more projections, walls or other surfaces. In certain embodiments of the invention, one or more damping materials is coupled to the matrix to provide a damping system for the reservoir. In some embodiments, the reservoir (optionally including a reservoir connector and a damping system) may be configured to be removable and replaceable with respect to the housing. In other embodiments, the reservoir may be secured to the housing in a manner intended to inhibit removal of the reservoir from the housing. Adhesive materials may be employed to adhere a surface of the reservoir to an engagement element, damping element or other structure on the housing. In further embodiments, multiple layers of adhesive material, alternating with multiple layers of damping layer material may be coupled to the reservoir, to allow the reservoir to be secured to, removed from and then, again, secured to the base or replaced with another reservoir to secure to the base, for reservoir replacement, refilling, or other service.

In certain embodiments of the invention, the fluid medication reservoir may be formed unitarily with a reservoir retaining portion of a housing, for example, as a hollow chamber provided within the housing. In such embodiments, the hollow interior of the reservoir retaining portion may be coated or lined with a suitable damping material and/or metal, plastic, plastic TOPAS (trademark of Ticona, a subsidiary of Celanese Corporation), ceramic, glass, composite material or the like. Alternatively, or in addition, the retaining portion itself may be made of a suitable metal, plastic, plastic, TOPAS (trademark of Ticona, a subsidiary of Celanese Corporation), ceramic, glass, composite material or the like.

In typical embodiments of the invention, the reservoir has an outlet through which the infusion medium contained within the interior of the reservoir may be communicated out of the reservoir. The outlet is open to the interior of the reservoir and may include one or more fluid conduits for connecting a fluid conduit in fluid flow communication with an outlet port. In this context, such fluid conduits which can hold therapeutic agents such as polypeptides can be operably coupled to a damping system as disclosed herein. The connection structure may include any suitable connection structure that may be selectively (or, in some embodiments, permanently) connected to a reservoir to provide fluid flow communication with the interior of the reservoir. For example, the connection structure may function with a pierceable septum located within the outlet port of the reservoir. In such embodiments, the connection structure may include a typical Luer-type connector having a cap structure for receiving an outlet port of the reservoir and a hollow needle for piercing the septum in the outlet port of the reservoir (such as, but not limited to, the reservoir connector 86 described in U.S. patent application Ser. No. 60/839,840, filed Aug. 23, 2006, titled “Infusion Medium Delivery Device), which is incorporated herein by reference, in its entirety. Further examples of needle/septum connectors can be found in U.S. patent application Ser. No. 10/328,393 filed Dec. 22, 2003, and entitled “Reservoir Connector,”. In other alternatives, non-septum connectors such as Luer locks, or the like may be used. The conduit may include any suitable structure that provides a fluid flow path, such as, but not limited to a tube-shaped conduit made of any suitable material, including, but not limited to a damping material such as a silicone or other plastic, as well as metals, ceramics or composite materials.

Therapeutic polypeptide formulations can be tailored for use with the damping systems disclosed herein. Therapeutic solutions or suspensions used for parenteral, intradermal, or subcutaneous application can include the following components: an agent selected for its ability to inhibit the aggregation of a therapeutic polypeptide (e.g. a Genapol®), a sterile diluent such as water for injection, saline solution; fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as EDTA; buffers such as acetates, citrates or phosphates and agents for the adjustment of tonicity such as sodium chloride or dextrose.

Therapeutic compositions of cytokines such as insulin and interferon-α can be prepared by mixing the desired cytokine having the appropriate degree of purity with aggregation inhibiting agents, pharmaceutically acceptable carriers, excipients, or stabilizers in the form of lyophilized formulations, aqueous solutions or aqueous suspensions (see, e.g. Remington: The Science and Practice of Pharmacy Lippincott Williams & Wilkins; 21 edition (2005), and Ansel's Pharmaceutical Dosage Forms and Drug Delivery Systems Lippincott Williams & Wilkins; 8th edition (2004)). For example, pharmaceutical compositions of pegylated interferon alpha-suitable for parenteral administration may be formulated with a suitable buffer, e.g., Tris-HCl, acetate or phosphate such as dibasic sodium phosphate/monobasic sodium phosphate buffer, and pharmaceutically acceptable excipients (e.g., sucrose), carriers (e.g. human plasma albumin), toxicity agents (e.g. NaCl), preservatives (e.g. thimerosol, cresol or benylalcohol), and surfactants (e.g. Tween or polysorabates) in sterile water for injection. Acceptable carriers, excipients, or stabilizers are typically nontoxic to recipients at the dosages and concentrations employed, and include buffers such as Tris, HEPES, PIPES, phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; sugars such as sucrose, mannitol, trehalose or sorbitol; salt-forming counter-ions such as sodium; and/or non-ionic surfactants such as TWEEN™, PLURONICS™ or polyethylene glycol (PEG). Solutions or suspensions used for administering a cytokine can include the following components: a sterile diluent such as water for injection, saline solution; fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as EDTA; buffers such as acetates, citrates or phosphates and agents for the adjustment of tonicity such as sodium chloride or dextrose.

### EXAMPLE 1

### Illustrative Embodiments of the Invention

This Example provides illustrative non-limiting systems and methods that allow one to collect and evaluate baseline data detailing the forces and temperatures experienced by a fluid in an external pump during one week of normal wear. The pump is the MiniMed 407C, which is commonly used for the administration of insulin in the treatment of diabetes, specifically Type 1. Forces were measured using a system of three accelerometers (one each in the X, Y, and Z directions) and the Ambulatory Data Recorder II (ADRII) system developed by the Neurological Division of Medtronic, Inc. (see, e.g., WO/2007/064642). The temperature of the solution in the pump reservoir was concurrently monitored using a small K-type wire thermocouple and data collector designed by Omega Engineering, Inc.

### Materials and Methods

### Temperature Recording Devices

The temperature of water within the fluid medication reservoir of a MiniMed 407C commercial insulin pump was detected using a K type, 30-gauge thermocouple wire obtained from Omega Engineering, Inc. (model # SC-TT-K-36-36) (FIG. 5, B and C). The needle-like portion of the reservoir that would normally connect the pump to the actual injection apparatus was removed, and the thermocouple wire was fed through the resulting hole so that the end of the wire extended into the center of the reservoir. Thermocouples were linked to a small data logger (Omega, model #OM-CP-TC4000) (FIG. 5, A and C). Both thermocouple temperature and ambient temperature were recorded by the logger every 60 seconds. Software designed by Omega to accompany the data logger enabled convenient extraction and visualization of collected data points.

### Force Recording Devices

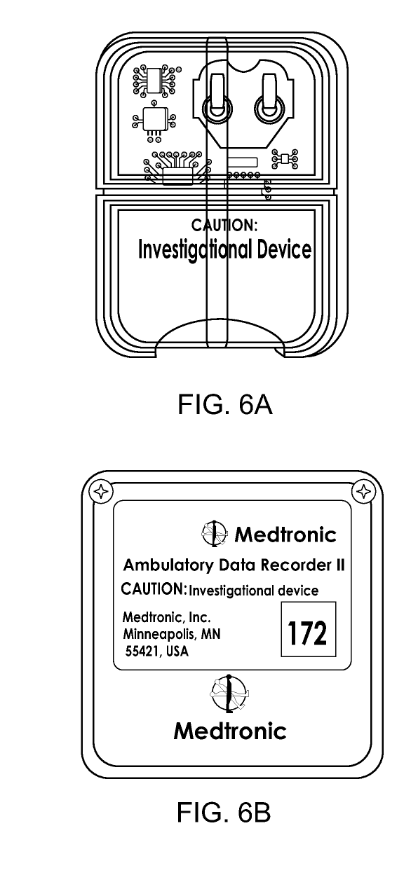

The forces exerted on the pump were measured and recorded by the Ambulatory Data Recorder II (ADRII) system developed by the Neurological division of Medtronic, Inc. This two-part system includes a small unit containing three accelerometers, one each in the X, Y, and Z directions relative to the accelerometer itself (FIG. 6A) and a data recorder that communicates wirelessly with the accelerometer component (FIG. 6B). The orientation of the ADRII relative to the pump is constant, but the orientation of the pump to the ground is not; forces experienced while riding a bus will be in a different than those experienced while jogging or rock climbing. Variations in body shape change the angle with the ground from participant to participant. One of the strengths of the MiniMed pump is its ability to be worn on the belt at any angle that is comfortable for a patient. The data generated reflected this flexibility, and the actual X, Y, and Z directions varied based on how the participant wore the pump. Measurements were taken with a frequency of 50 Hz. The recorder stored data to a MicroSD card in binary files that could be directly accessed by a computer. A Perl script provided by Medtronic was applied to parse the data from the *.bin files into a readable, usable form (comma separated values, or *.csv). FIG. 4 provides a graph of raw and reduced data obtained from accelerometers coupled to medication infusion pumps. In this figure the blue, green, and red plots are accelerometer data for the x, y, and z directions respectively, while the aqua color plot is the resultant acceleration magnitude calculated from the other three. The y-axis represents acceleration magnitude and the x-axis is the sample number (proportional to time).

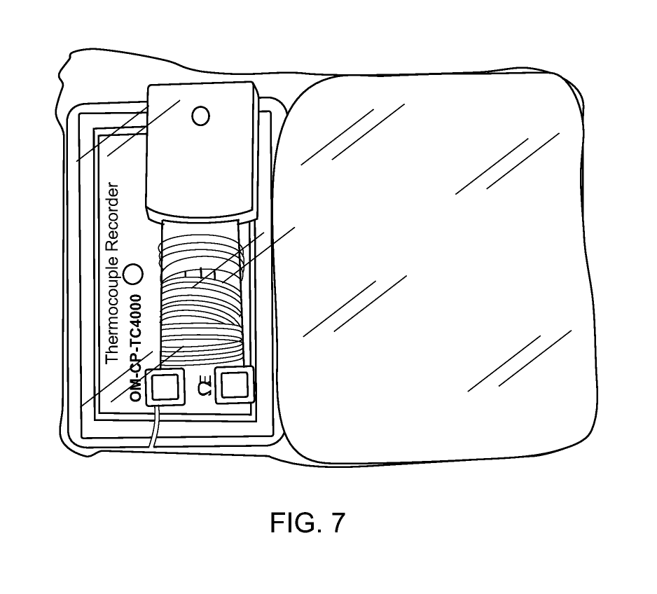

The insulin pump, thermocouple system, and ADRII accelerometer unit were coupled together into a compact, wearable device with a belt clip (FIG. 7). Components were wrapped in plastic wrap to protect them from tape residue and taped together firmly with clear packing tape to prevent movement in relation to each other. The ADRII data recorder was placed in a separate pouch with belt clip to facilitate convenient wearing. Three composite systems were prepared, allowing data sets to be collected from three separate participants simultaneously.

### User Protocol

Users were recruited to participate based on their interest, willingness to cooperate with trial expectations, and agreement to specific participation requirements. Each participant was expected to wear the composite device on their hip during all waking hours for one week, excluding bathing, swimming, or other activities such as contact sports which may have damaged the system and proved harmful to the student.

### Results

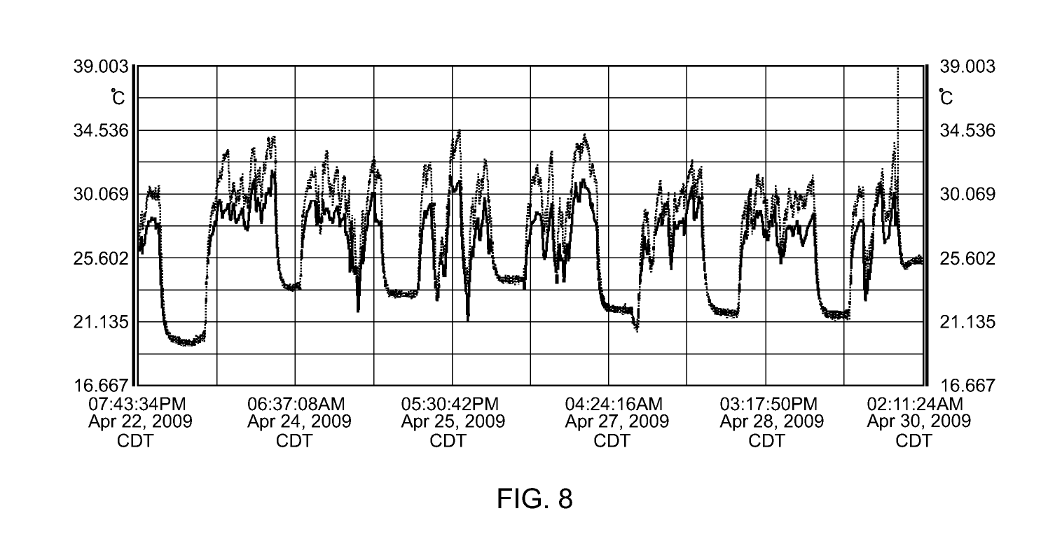

Six data sets were collected over a two week period. These were organized and stored on a USB flash drive. Temperature data was easily visualized using the Omega 2.00.73 software (FIG. 8).

Force data was parsed from the binary (*.bin) files into comma-separated values (*.csv) files, but required a program able to handle large data sets, such as Matlab, to be effectively visualized (each participant generated approximately 90,720,000 data points over the week). Data recorded by the ADRII recorder was raw 10 bit data from the sensors. Accelerometer data is generally considered in terms of “g”, or the acceleration due to gravity on earth. 1 g is defined as the standard acceleration due to gravity at sea level, about 9.81 m/s². Variations from this are then measured in multiples of g. To convert the 10 bit data to g's, the minimum and maximum values for each individual sensor were considered. The average of these was equivalent to 0 g for that particular sensor. The maximum g value and average g value were used to find the ratio to convert from bits to g's for that sensor.

This Example provides illustrative effective systems for the concurrent measurement of temperatures and forces experienced by an external drug pump. Those of skill in the art will understand that there are a variety of permutations of the disclosed methods, materials, systems, kits etc. and that one can mix and match the various elements disclosed herein to generate a variety of embodiments. Throughout this application, various journal articles, patents, patent applications, and other publications etc. are referenced to provide illustrations of the state of the art. The present invention is not to be limited in scope by the embodiments disclosed herein, which are intended as single illustrations of individual aspects of the invention, and any that are functionally equivalent are within the scope of the invention. Further, even though the invention herein has been described with reference to particular examples and embodiments, it is to be understood that these examples and embodiments are merely illustrative of the principles and applications of the present invention. It is therefore to be understood that numerous modifications may be made to the illustrative embodiments and that other arrangements may be devised without departing from the spirit and scope of the present invention as defined by the following claims. Publications describing aspects of this technology include for example, U.S. Pat. Appln. Nos. 2008/0051716; 2008/0009824; 2008/102119; and 2008/0097375 and U.S. Pat. Nos. 7,658,734 and 7,288,085; U.S. Pat. Appln. Nos. 2005/0063949 and 2007/0077225; U.S. Pat. Nos. 6,172,046; 6,245,740; 5,824,784; 5,372,808; 5,980,884; published international patent applications WO 96/21468; WO 96/11953; Torre et al. (2001) J. Med. Virol. 64:455-459; Bekkering et al. (2001) J. Hepatol. 34:435-440; Zeuzem et al. (2001) Gastroenterol. 120:1438-1447; Zeuzem (1999) J. Hepatol. 31:61-64; Keeffe and Hollinger (1997) Hepatol. 26:101S-107S; Wills (1990) Clin. Pharmacokinet. 19:390-399; Heathcote et al. (2000) New Engl. J. Med. 343:1673-1680; Husa and Husova (2001) Bratisl. Lek. Listy 102:248-252; Glue et al. (2000) Clin. Pharmacol. 68:556-567; Bailon et al. (2001) Bioconj. Chem. 12:195-202; and Neumann et al. (2001) Science 282:103; Zalipsky (1995) Adv. Drug Delivery Reviews S. 16, 157-182; Mann et al. (2001) Lancet 358:958-965; Zeuzem et al. (2000) New Engl. J. Med. 343:1666-1672; U.S. Pat. Nos. 5,985,265; 5,908,121; 6,177,074; 5,985,263; 5,711,944; 5,382,657; and 5,908,121; Osborn et al. (2002) J. Pharmacol. Exp. Therap. 303:540-548; Sheppard et al. (2003) Nat. Immunol. 4:63-68; Chang et al. (1999) Nat. Biotechnol. 17:793-797; Adolf (1995) Multiple Sclerosis 1 Suppl. 1:S44-S47. Various modifications to the models and methods of the invention, in addition to those described herein, will become apparent to those skilled in the art from the foregoing description and teachings, and are similarly intended to fall within the scope of the invention. Such modifications or other embodiments can be practiced without departing from the true scope and spirit of the invention. However, the invention is only limited by the scope of the appended claims. All numbers recited in the specification and associated claims that refer to values that can be numerically characterized (e.g. the duration of a treatment, the concentration of a therapeutic compound etc.) can be modified by the term “about”.

1. A medication infusion pump comprising:
a housing;
a fluid medication reservoir disposed within the housing and adapted to store a medication to be administered to a user; and
a damping system adapted to inhibit agitation of a medication stored within the fluid medication reservoir, wherein the damping system:
comprises one or more layers of a damping material disposed between the housing and the medication reservoir; and
envelopes the fluid medication reservoir.

2. The medication infusion pump of claim 1, wherein the medication infusion pump comprises a continuous infusion pump having dimensions smaller than 15×15 centimeters.

3. The medication infusion pump of claim 1, wherein the damping system is disposed on a reservoir connector comprising:
a cap for coupling the fluid medication reservoir to a fluid conduit;
a conduit cavity disposed in the cap and adapted to secure the fluid conduit to the cap;
a housing engagement member adapted to engage the housing; and
wherein the reservoir connector is adapted to be releasably secured within the housing.

4. The medication infusion pump of claim 1, wherein the fluid medication reservoir comprises a deformable container adapted to inhibit the formation of void spaces within the container as fluid is removed from the container.

5. The medication infusion pump of claim 1, wherein the damping system further comprises one or more vibration absorbing materials disposed over the housing, wherein the vibration absorbing materials are adapted to decrease the magnitude of accelerative force imparted on a medication disposed within the fluid medication reservoir.

6. The medication infusion pump of claim 5, wherein the vibration absorbing material comprises a rubber, a silicone, a polyurethane, a polyethylene, a polypropylene, a non conjugated diene, or combinations thereof.

7. The medication infusion pump of claim 1, wherein the damping system comprises a fluid reservoir mount including one or more layers of a vibration absorbing material in operable contact with the medication reservoir.

8. The medication infusion pump of claim 1, further comprising a polypeptide medication disposed within the fluid medication reservoir, wherein the fluid medication is combined with an agent selected to inhibit the aggregation of the polypeptide.

9. The medication infusion pump of claim 1, wherein the damping system comprises a gimbal.

10. The continuous infusion pump of claim 9, wherein the pump is operably coupled to an interface that facilitates its attachment to a user, wherein the interface comprises a clip, a strap, a snap, a clamp or an adhesive strip.

11. A medication infusion system comprising:
infusion set tubing;
a medication infusion pump comprising:
a housing;
a fluid medication reservoir disposed within the housing and adapted to store a medication to be administered to a patient;
a damping system adapted to inhibit agitation of a fluid stored within the fluid medication reservoir wherein the damping system:
comprises one or more layers of a damping material disposed between the housing and the medication reservoir; and
surrounds the fluid medication reservoir;
a program memory;
a processor for reading and executing the instructions contained in the program memory;
a motor adapted to deliver a fluid medication to a user; and
an interface that facilitates attachment of the pump to the user; and/or
a reservoir connector that facilitates fluid medication reservoir insertion into and removal from the housing of the medication infusion pump.

12. The medication infusion system of claim 11, wherein the damping system further comprises:
one or more layers of a vibration absorbing material disposed on the housing, wherein at least one layer comprises a viscoelastic material for reducing accelerations to the pump that result from movement of the housing; and
at least one layer comprises a viscoelastic material for reducing accelerations to the pump that result from movement of the housing.

13. The medication infusion system of claim 12, wherein the vibration absorbing material is further selected for an ability to insulate the fluid medication reservoir from fluctuations in temperature experienced by the medication infusion pump.

14. The medication infusion system of claim 11, wherein:
the damping system comprises a motion or temperature sensor coupled to an alarm that is activated when the sensor detects a mechanical stress or temperature that exceeds a critical threshold value;
the damping system includes a fluid reservoir mount comprising a gimbal; and/or
the interface that facilitates attachment of the pump to the user comprises a gimbal.

15. The medication infusion system of claim 11, wherein:
the fluid medication reservoir comprises an insulin or an interferon; and
the pump is a continuous infusion pump having dimensions smaller than 15×15 centimeters.

16. A method of inhibiting aggregation of a polypeptide contained in a fluid medication reservoir within a medication infusion pump, wherein the pump is used to deliver a fluid medication to a user, the method comprising using a medication infusion pump comprising:
a housing;
a fluid medication reservoir disposed within the housing and adapted to store a medication to be administered to a patient; and
a damping system adapted to inhibit agitation of a fluid stored within the fluid medication reservoir, wherein the damping system:
comprises one or more layers of a damping material disposed between the housing and the medication reservoir; and
envelopes the fluid medication reservoir;
and wherein the inhibition of agitation inhibits the aggregation of a polypeptide contained in the fluid medication reservoir.

17. The method of claim 16, wherein the damping system further comprises one or more vibration absorbing materials disposed over the housing, wherein the vibration absorbing materials are adapted to decrease the magnitude of accelerative force imparted on a medication disposed within the fluid medication reservoir.

18. The method of claim 16, wherein the damping system comprises a fluid reservoir mount including one or more layers of a vibration absorbing material.

19. The method of claim 18, wherein the fluid reservoir mount comprises:
a cap for coupling the fluid medication reservoir to a fluid conduit;
a conduit cavity disposed in the cap and adapted to secure the fluid conduit to the cap;
a housing engagement member adapted to engage the housing; and
a tab disposed on the cap to provide a surface for a user to grip and twist the cap so as to engage the medication pump housing upon rotation of the cap.

20. The method of claim 16, further comprising using a fluid medication reservoir comprising a deformable container adapted to inhibit the formation of void spaces within the container as fluid is removed from the container.
